(19) United States
(12) Patent Application Publication
(10) Pub. No.: US 2026/0055075 A1
(43) Pub. Date: Feb. 26, 2026

(54) STABLE POLYMORPH OF R-MDMA HCL

(71) Applicant: EmpathBio, Inc., New York, NY (US)

(72) Inventors: Majed FAWAZ, Foxborough, MA (US); Nicholas MORRA, Ontario (CA)

(21) Appl. No.: 19/366,168

(22) Filed: Oct. 22, 2025

Related U.S. Application Data
(63) Continuation of application No. 18/116,195, filed on Mar. 1, 2023, now Pat. No. 12,492,178.
Application No. 18/116,195 is a continuation-in-part of application No. 17/901,504, filed on Sep. 1, 2022, now Pat. No. 11,993,577.
(60) Provisional application No. 63/239,853, filed on Sep. 1, 2021.

(51) Int. Cl. C07D 317/58

(52) U.S. Cl. CPC C07D 317/58; C07B 2200/13

(57) ABSTRACT

Provided herein is a process for the preparation of (R)-3,4 methylenedioxymethamphetamine HCl Form 1.

### CROSS-REFERENCE TO RELATED APPLICATIONS

The present application is a continuation of U.S. patent application Ser. No. 18/116,195 filed Mar. 1, 2023, which is a continuation-in-part of U.S. patent application Ser. No. 17/901,504, filed Sep. 1, 2022, now U.S. Pat. No. 11,993,577, which claims priority to U.S. Provisional Patent Application No. 63/239,853 filed Sep. 1, 2021, all of which are hereby incorporated by reference in their entireties.

### BACKGROUND OF THE INVENTION

3,4-Methylenedioxymethamphetamine (MDMA) is a substituted amphetamine class, and its free base form is a colorless oil, insoluble in water. The most common salt of MDMA is the hydrochloride salt, which appears as a white or off-white powder or crystal and is soluble in water. The polymorphs of the hydrochloride salt of racemic MDMA have been described.

Synthesis of R- and S-MDMA HCl has been described by Huot et al., “Characterization of 3,4-Methylenedioxymethamphetamine (MDMA) Enantiomers In Vitro and in the MPTP-Lesioned Primate: R-MDMA Reduces Severity of Dyskinesia, Whereas S-MDMA Extends Duration of ON-Time,” J. of Neuroscience, 31(19):7190-7198 (2011). The synthesis involves an enantiospecific and high-yielding ring opening of enantiopure aziridines 2 and 3 by the Grignard reagent 1, followed by subsequent methylation and detosylation to yield R- and S-MDMA:

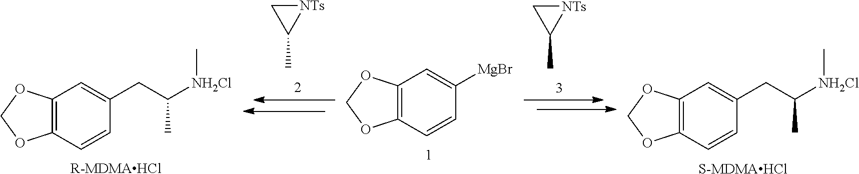

Although existing reports describe polymorphs of the hydrochloride salt of racemic MDMA, there remains a need to identify stable polymorphic crystalline forms of R-MDMA HCl and produce compositions comprising stable crystalline forms of R-MDMA HCl, the compound of formula (I):

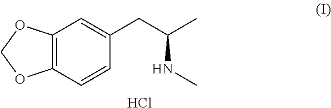

### SUMMARY OF THE INVENTION

In an aspect, the present disclosure provides a process for the preparation of 3,4-methylenedioxymethamphetamine (MDMA).

In some embodiments, the present disclosure provides a process for the preparation of Form 1 enantiopure (R)-3,4-methylenedioxymethamphetamine (MDMA) HCl salt.

In embodiments, R-MDMA HCl Form I is characterized by an X-ray Powder Diffraction (XPRD) pattern substantially similar to 204 in FIG. 2A and FIG. 2B. In Embodiments, MDMA HCl Form I is characterized by peaks in an XPRD pattern at 17.5±0.2, 24.8±0.2, 15.8±0.2, 20.5±0.2, 14.0±0.2, 26.7±0.2, 29.0±0.2, 37.9±0.2, 7.9±0.2° 2θ.

In embodiments, R-MDMA HCl Form I exhibits a monoclinic crystal system with a space group of P21 and cell parameters of a=7.17±0.05 Å, b=7.61±0.05 Å, c=11.75±0.05 Å, α=90±1°, β=107±1°, γ=90±1°, V=612±5 Å³/cell.

In embodiments, R-MDMA HCl Form I exhibits a solubility of >110 mg/mL in water.

In embodiments, R-MDMA HCl Form I exhibits a Differential Scanning calorimetry (DSC) thermogram comprising an endotherm peak at 186° C. with an onset at 185° C.

In embodiments, R-MDMA HCl Form I exhibits 0.2±0.1% weight loss between 52-231° C. as measured by thermogravimetric (TGA) analysis.

In embodiments, R-MDMA HCl Form I exhibits 5.28±0.02% weight gain from 5-95% relative humidity (RH) and a 5.35±0.02% weight loss from 95-5% RH at 10% RH increments under a nitrogen purge as measured by Dynamic Vapor Sorption (DVS).

In embodiments, R-MDMA HCl Form I exhibits physical stability upon stressing at 97% RH for 6 days.

In some embodiments, the present disclosure provides: a process for the preparation of (R)-3,4-methylenedioxymethamphetamine HCl Form 1, the process comprising:
i) reacting an organometallic reagent prepared from a compound of Formula (I):

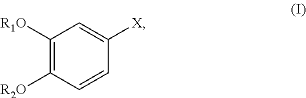

with a compound of Formula (IIa):

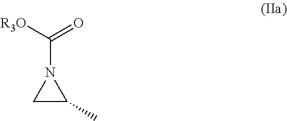

wherein R₃ is alkyl; and
ii) converting the product of step i) to (R)-3,4-methylenedioxymethamphetamine, wherein X is a halogen; R₁ is a protecting group, R₂ is a protecting group or R₁ and R₂ together with the atoms to which they are attached form a 5-membered heterocycle.

In some embodiments, X is preferably bromine.

In some embodiments, the process provides (R)-3,4-methylenedioxymethamphetamine HCl Form 1 in an enantiomeric excess of a least 99.5%.

### BRIEF DESCRIPTION OF THE FIGURES

FIG. 1 shows a structure of R-MDMA HCl;

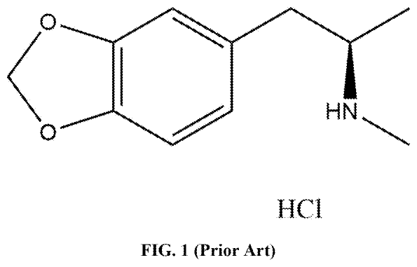

FIG. 2A shows an indexed XRPD diffractogram of a sample of synthesized R-MDMA HCl Form 1 overlayed a sample of R-MDMA HCl reported in literature;

FIG. 2B shows the indexed XRPD diffractogram of the sample of the synthesized R-MDMA HCl Form 1 overlayed a calculated XRPD from single crystal data of the R-MDMA sample;

FIG. 2C shows an atomic displacement ellipsoid drawing of R-MDMA HCl Form 1;

FIG. 3A shows an ¹H NMR parameter table for a sample of the synthesized R-MDMA HCl Form 1;

FIG. 3B shows an ¹H NMR spectrum for the sample of the synthesized R-MDMA HCl Form 1;

FIG. 4 shows a DSC and TGA thermogram of a sample of the synthesized R-MDMA HCl From 1;

FIG. 5 shows a DVS isotherm of a sample of the synthesized R-MDMA HCl Form 1;

FIG. 6 shows a microscope image of a sample of the synthesized R-MDMA HCl Form 1;

FIG. 7 shows a cycling DSC thermogram of a sample of the synthesized R-MDMA HCl Form 1 with a rate of 10K/min on all steps; and

FIG. 8 shows a cycling DSC thermogram of a sample of the synthesized R-MDMA HCl Form 1 with a rate of 200K/min on the cooling step.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “treating” as used herein with regard to a patient or subject, refers to improving at least one symptom of the patient's or subject's disorder. In some embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient or subject in need thereof.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

The term “substituted” used herein means any of the groups described herein (e.g., alkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity can be specified by inference. For example, the compound CH₃—R^{3X}, wherein R^{3X} is H or “ [symbol] ” infers that when R^{3X} is “XY”, the point of attachment bond is the same bond as the bond by which R^{3X} is depicted as being bonded to CH₃.

### Crystalline Forms

In some embodiments of the present disclosure, a crystalline form of R-MDMA HCl (e.g., Form 1) is provided. The crystallin form may be characterized by a XRPD diffraction pattern.

### Compositions

In some embodiments of the present disclosure, a pharmaceutical composition comprising in a therapeutically effective amounts a stable polymorphic form of R-MDMA HCl (e.g., Form 1) and a pharmaceutically acceptable excipient is provided.

The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

### Methods

In an aspect, the methods described herein provide high purity, 3,4-methylenedioxymethamphetamine (MDMA) in a high yielding 2-step process, starting from readily available and inexpensive starting materials (e.g., 5-bromobenzo[d][1,3]dioxole (3) and alaninol). In an aspect, the disclosure provides a process for preparation of racemic 3,4-methylenedioxymethamphetamine or a pharmaceutically acceptable salt thereof.

In an aspect, the disclosure provides a process for the preparation of (R)- or(S)-3,4-methylenedioxymethamphetamine, or a pharmaceutically acceptable salt thereof.

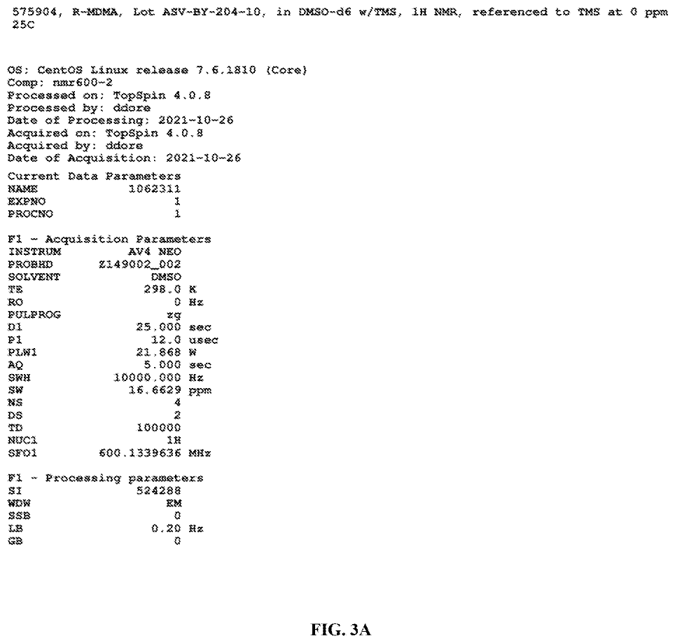

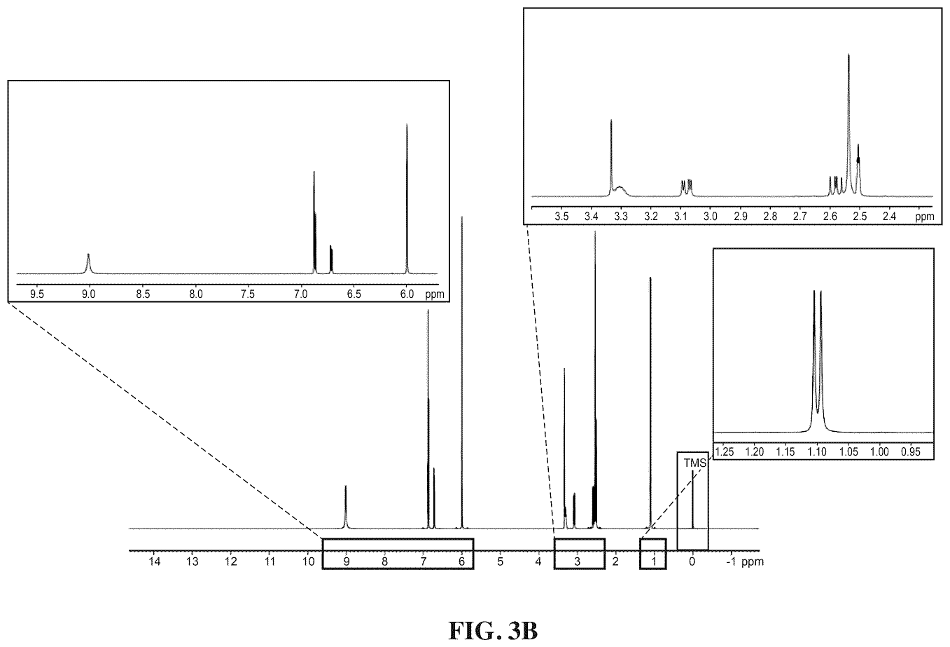

In embodiments, the methods described herein provide high purity, enantiopure (R) or (S) 3,4-methylenedioxymethamphetamine (MDMA) in a high yielding 2-step process, starting from readily available and inexpensive starting materials (e.g., 5-bromobenzo[d][1,3]dioxole (3) and D-alaninol (1)). By starting with a chiral pool starting materials, such as (D-alaniol (1)), enantiopure (R)-MDMA can be prepared without the need for expensive and wasteful chiral ligands, chiral auxiliaries, or diastereomeric salt resolutions, and provide MDMA with higher optical purity (e.g., 99.5% ee or greater, or 99.9% ee) than other routes, which give lower selectivity and require enantiomeric enrichment by purification/crystallization. By installing a Boc group as both a nitrogen protecting group and a masked methyl equivalent, the need for a methylation reaction is avoided, which often uses GTI alkylating reagents like methyl iodide or dimethylsulfate. It also cleanly installs the single methyl group and avoids the potential of over-alkylation and provides enantioenriched (R)-MDMA in a streamlined process, providing significant improvements on prior syntheses which are more complex and/or require synthetic longer routes see e.g., ACS Chem Neurosci. 2018 Oct. 17; 9(10): 2408-2427, FIG. 3. The unnatural enantiomer of (1) (L-alaninol) can be used by the same process to prepare (S)-MDMA.

In embodiments, the methods described herein provide high purity, enantiopure (R) 3,4-methylenedioxymethamphetamine (MDMA) in single anhydrous crystal form designated Form 1.

### Preparation of 3,4-methylenedioxymethamphetamine

In an aspect, the disclosure provides a process for the preparation of 3,4-methylenedioxymethamphetamine, or a pharmaceutically acceptable salt thereof, the process comprising:
i) reacting an organometallic reagent prepared from a compound of Formula (I):

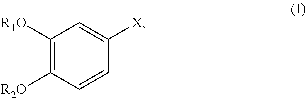

with a compound of Formula (IIb):

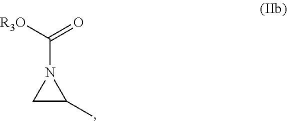

wherein R₃ is alkyl; and
ii) converting the product of step i) to 3,4-methylenedioxymethamphetamine, wherein X is a halogen; R₁ is a protecting group, R₂ is a protecting group or R₁ and R₂ together with the atoms to which they are attached form a 5-membered heterocycle. In embodiments, the compound of Formula (IIb) is a racemate.

In embodiments of the processes provided herein for the preparation of 3,4-methylenedioxymethamphetamine, the process comprises i) preparing an organometallic reagent from a compound of Formula (I):

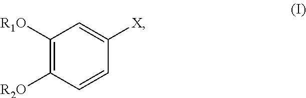

wherein X is a halogen; R₁ is a protecting group, R₂ is a protecting group or R₁ and R₂ together with the atoms to which they are attached form a 5-membered heterocycle.

In some embodiments of any one of the processes for the preparation of 3,4-methylenedioxymethamphetamine described herein, R₁ and R₂ together with the atoms to which they are attached form a 5-membered heterocycle.

In some embodiments of the process for the preparation of 3,4-methylenedioxymethamphetamine, the compound of Formula (I) is a compound of Formula (Ia):

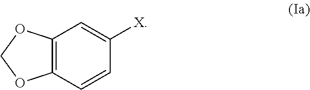

In embodiments of the compounds of Formula (I) or (Ia), X is Cl, Br, or I. In some embodiments, X is Cl. In some embodiments, X is Br. In some embodiments, X is I.

In some embodiments of the process for the preparation of 3,4-methylenedioxymethamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, or 2-methyltetrahydrofuran. In some embodiments, the solvent is heated e.g., to 50-70° C., 60-70° C., or 60-66° C. In some embodiments, the solvent is heated to reflux. In some embodiments, the solvent is THF and the THF is heated to reflux.

In some embodiments of the process for the preparation of 3,4-methylenedioxymethamphetamine, step i) further comprises adding a copper (I) salt (e.g., CuI, CuCl, or CuBr.SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyamphetamine.

In embodiments of the process for the preparation of 3,4-methylenedioxymethamphetamine, step i) comprises reacting the organometallic reagent of step i) with a compound of Formula (IIb):

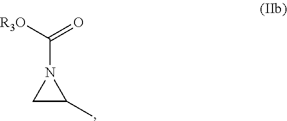

wherein R₃ is alkyl. In some embodiments, step i) comprises adding a compound of Formula (IIb) to the cooled solution of organometallic reagent.

In some embodiments of the process for the preparation of 3,4-methylenedioxymethamphetamine, R₃ is C₁₋₆ alkyl. In some embodiments, R₃ is C₁₋₄ alkyl. In some embodiments, R₃ is tert-butyl.

In some embodiments of the process for the preparation of 3,4-methylenedioxymethamphetamine, the product of step i) is a compound of Formula (IIIb):

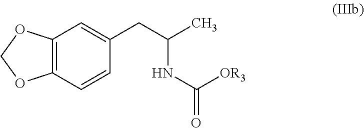

wherein R₃ is defined herein.

In some embodiments of the process for the preparation of 3,4-methylenedioxymethamphetamine, the product of step i) is a compound of Formula (IIIc):

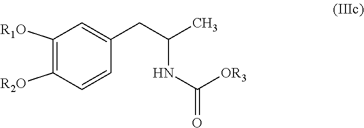

wherein R₁, R₂, and R₃ are defined herein.

In some embodiments of the process for the preparation of 3,4-methylenedioxymethamphetamine, step ii) comprises reacting a group of Formula (IVb) with a reducing agent to provide a group of Formula (Vb), or a pharmaceutically acceptable salt thereof:

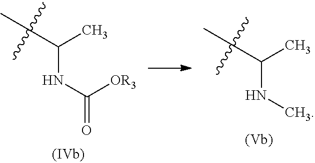

In some embodiments, step ii) comprises reacting a group of Formula (IVb) with a reducing agent to provide a group of Formula (Vb), or a pharmaceutically acceptable salt thereof in the presence of a solvent, for example an ether solvent such as tetrahydrofuran, diethyl ether, 2-methyltetrahydrofuran. In some embodiments, the solvent is heated. In some embodiments, the solvent is refluxing THF.

In some embodiments of the process for the preparation of 3,4-methylenedioxymethamphetamine, the reducing agent in step ii) is a hydride reducing agent. In some embodiments, the reducing agent in step ii) is lithium aluminum hydride.

In some embodiments of the process for the preparation of 3,4-methylenedioxymethamphetamine, the process provides 3,4-methylenedioxymethamphetamine, or a pharmaceutically acceptable salt thereof in racemic form.

In some embodiments of the process for the preparation of 3,4-methylenedioxymethamphetamine, the 3,4-methylenedioxymethamphetamine, or a pharmaceutically acceptable salt thereof has a chemical purity of greater than 95%, greater than 98%, or greater than 99% by HPLC.

In some embodiments, the present disclosure provides 3,4-methylenedioxymethamphetamine, or a pharmaceutically acceptable salt prepared by a process described herein.

### Preparation of (S)-3,4-methylenedioxymethamphetamine

In an aspect, the disclosure provides a process for the preparation of (S)-3,4-methylenedioxymethamphetamine, or a pharmaceutically acceptable salt thereof, the process comprising:
i) reacting an organometallic reagent prepared from a compound of Formula (I):

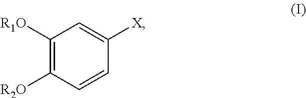

with a compound of Formula (II):

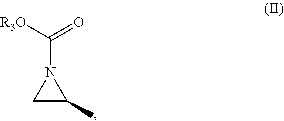

wherein R₃ is alkyl; and
ii) converting the product of step i) to (S)-3,4-methylenedioxymethamphetamine wherein X is a halogen; R₁ is a protecting group, R₂ is a protecting group or R₁ and R₂ together with the atoms to which they are attached form a 5-membered heterocycle.

In embodiments of the processes provided herein for the preparation of (S)-3,4-methylenedioxymethamphetamine, the process comprises i) preparing an organometallic reagent from a compound of Formula (I):

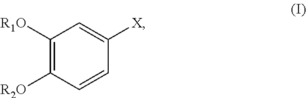

wherein X is a halogen; R₁ is a protecting group, R₂ is a protecting group or R₁ and R₂ together with the atoms to which they are attached form a 5-membered heterocycle.

In some embodiments of any one of the processes for the preparation of (S)-3,4-methylenedioxymethamphetamine described herein, R₁ and R₂ together with the atoms to which they are attached form a 5-membered heterocycle.

In some embodiments of the process for the preparation of (S)-3,4-methylenedioxymethamphetamine, the compound of Formula (I) is a compound of Formula (Ia):

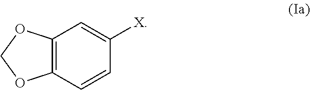

In embodiments of the compounds of Formula (I) or (Ia), X is Cl, Br, or I. In some embodiments, X is Cl. In some embodiments, X is Br. In some embodiments, X is I.

In some embodiments of the process for the preparation of (S)-3,4-methylenedioxymethamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, 2-methyltetrahydrofuran. In some embodiments, the solvent is heated e.g., to 50-70° C., or 60-70° C., or 60-66° C. In some embodiments, the solvent is heated to reflux. In some embodiments, the solvent is THF and the THF is heated to reflux.

In some embodiments of the process for the preparation of (S)-3,4-methylenedioxymethamphetamine, step i) further comprises adding a copper (I) salt (e.g., CuI, CuCl, CuBr. SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyaphetamine.

In embodiments of the process for the preparation of (S)-3,4-methylenedioxymethamphetamine, step i) further comprises reacting the organometallic reagent with a compound of Formula (II):

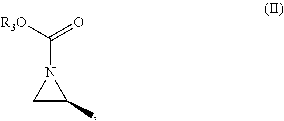

wherein R₃ is alkyl. In some embodiments, step i) comprises adding a compound of Formula (II) to the cooled solution of organometallic reagent.

In some embodiments of the process for the preparation of (S)-3,4-methylenedioxymethamphetamine, R₃ is C₁₋₆ alkyl. In some embodiments, R₃ is C₁₋₄ alkyl. R₃ is tert-butyl.

In some embodiments of the process for the preparation of (S)-3,4-methylenedioxymethamphetamine, the product of step ii) is a compound of Formula (III):

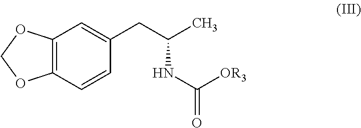

wherein R₃ is defined herein.

In some embodiments of the process for the preparation of (S)-3,4-methylenedioxymethamphetamine, the product of step i) is a compound of Formula (IIIa):

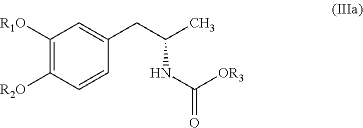

wherein R₁, R₂, and R₃ are defined herein.

In some embodiments of the process for the preparation of (S)-3,4-methylenedioxymethamphetamine, step ii) comprises reacting a group of Formula (IV) with a reducing agent to provide a group of Formula (V), or a pharmaceutically acceptable salt thereof:

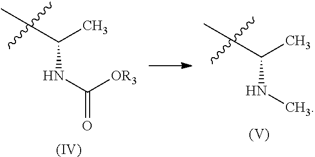

In some embodiments, step ii) comprises reacting a group of Formula (IV) with a reducing agent to provide a group of Formula (V), or a pharmaceutically acceptable salt thereof in the presence of a solvent, for example an ether solvent such as tetrahydrofuran, diethyl ether, 2-methyltetrahydrofuran. In some embodiments, the solvent is heated. In some embodiments, the solvent is refluxing THF.

In some embodiments of the process for the preparation of (S)-3,4-methylenedioxymethamphetamine, the reducing agent in step ii) is a hydride reducing agent. In some embodiments, the reducing agent in step ii) is lithium aluminum hydride.

In some embodiments of the process for the preparation of (S)-3,4-methylenedioxymethamphetamine, the process provides (S)-3,4-methylenedioxymethamphetamine, or a pharmaceutically acceptable salt thereof in substantially optically pure form. In some embodiments, the process for the preparation of (S)-3,4-methylenedioxymethamphetamine, the process provides (S)-3,4-methylenedioxymethamphetamine, or a pharmaceutically acceptable salt in an enantiomeric excess (ee) of about or at least about 55% ee, about or at least about 60% ee, about or at least about 65% ee, about or at least about 70% ee, about or at 75% ee, about or at least about 80% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all subranges and values therebetween. In some embodiments, the process for the preparation of (S)-3,4-methylenedioxymethamphetamine, the process provides (S)-3,4-methylenedioxymethamphetamine, or a pharmaceutically acceptable salt in an enantiomeric excess of a least 99.5%. In some embodiments, ee is measured by chiral HPLC.

In some embodiments of the process for the preparation of (S)-3,4-methylenedioxymethamphetamine, the (S)-3,4-methylenedioxymethamphetamine, or a pharmaceutically acceptable salt thereof has a chemical purity of greater than 95%, greater than 98%, or greater than 99% by HPLC.

In some embodiments, the present disclosure provides (S)-3,4-methylenedioxymethamphetamine, or a pharmaceutically acceptable salt prepared by a process described herein.

### Preparation of (R)-3,4-methylenedioxymethamphetamine

In an aspect, the disclosure provides a process or the preparation of (R)-3,4-methylenedioxymethamphetamine, or a pharmaceutically acceptable salt thereof, the method comprising:
i) reacting an organometallic reagent prepared from a compound of Formula (I):

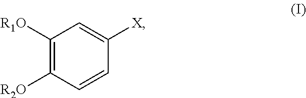

with a compound of Formula (IIa):

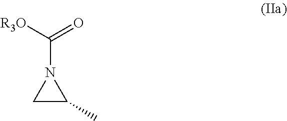

wherein R₃ is alkyl; and
ii) converting the product of step i) to (R)-3,4-methylenedioxymethamphetamine, wherein X is a halogen; R₁ is a protecting group, R₂ is a protecting group or R₁ and R₂ together with the atoms to which they are attached form a 5-membered heterocycle.

In embodiments of the processes provided herein for the preparation of (R)-3,4-methylenedioxymethamphetamine, the process comprises i) preparing an organometallic reagent from a compound of Formula (I):

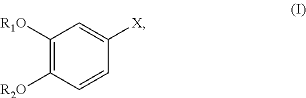

wherein X is a halogen; R₁ is a protecting group, R₂ is a protecting group or R₁ and R₂ together with the atoms to which they are attached form a 5-membered heterocycle.

In some embodiments of any one of the processes for the preparation of (R)-3,4-methylenedioxymethamphetamine described herein, R₁ and R₂ together with the atoms to which they are attached form a 5-membered heterocycle.

In some embodiments of the process for the preparation of (R)-3,4-methylenedioxymethamphetamine, the compound of Formula (I) is a compound of Formula (Ia):

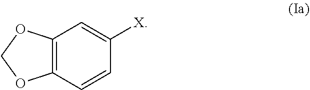

In embodiments of the compounds of Formula (I) or (Ia), X is Cl, Br, or I. In some embodiments, X is Cl. In some embodiments, X is Br. In some embodiments, X is I.

In some embodiments of the process for the preparation of (R)-3,4-methylenedioxymethamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, 2-methyltetrahydrofuran. In some embodiments, the solvent is heated e.g., to 50-70° C. In some embodiments, the solvent is THF and the THF is heated to reflux.

In some embodiments of the process for the preparation of (R)-3,4-methylenedioxymethamphetamine, step i) further comprises adding a copper (I) salt (e.g., CuI, CuCl, CuBr. SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyamphetamine.

In some embodiments of the process for the preparation of (R)-3,4-methylenedioxymethamphetamine comprises reacting the organometallic reagent with a compound of Formula (IIa):

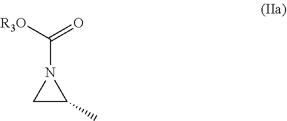

wherein R₃ is alkyl. In some embodiments, step i) comprises adding a compound of Formula (IIa) to the cooled solution of organometallic reagent.

In some embodiments of the process for the preparation of (R)-3,4-methylenedioxymethamphetamine, R₃ is C₁₋₆ alkyl. In some embodiments, R₃ is C₁₋₄ alkyl. R₃ is tert-butyl.

In some embodiments of the process for the preparation of (R)-3,4-methylenedioxymethamphetamine, the product of step i) is a compound of Formula (IIIb):

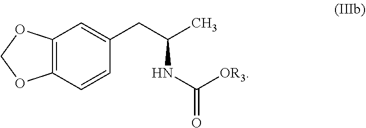

In some embodiments of the process for the preparation of (R)-3,4-methylenedioxymethamphetamine, the product of step i) is a compound of Formula (IIIb′):

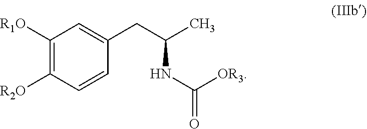

In some embodiments of the process for the preparation of (R)-3,4-methylenedioxymethamphetamine, step ii) comprises reacting a group of Formula (IVa) with a reducing agent to provide a group of Formula (Va), or a pharmaceutically acceptable salt thereof:

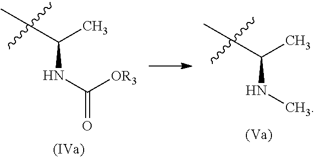

In some embodiments, step ii) comprises reacting a group of Formula (IV) with a reducing agent to provide a group of Formula (V), or a pharmaceutically acceptable salt thereof in the presence of a solvent, for example an ether solvent such as tetrahydrofuran, diethyl ether, 2-methyltetrahydrofuran.

In some embodiments of the process for the preparation of (R)-3,4-methylenedioxymethamphetamine, the reducing agent in step ii) is a hydride reducing agent. In some embodiments, the reducing agent in step ii) is lithium aluminum hydride.

In some embodiments of the process for the preparation of (R)-3,4-methylenedioxymethamphetamine, the process provides (R)-3,4-methylenedioxymethamphetamine, or a pharmaceutically acceptable salt thereof in substantially optically pure form. In some embodiments, the process for the preparation of (R)-3,4-methylenedioxymethamphetamine, the process provides (R)-3,4-methylenedioxymethamphetamine, or a pharmaceutically acceptable salt in an enantiomeric excess (ee) of about or at least about 55% ee, about or at least about 60% ee, about or at least about 65% ee, about or at least about 70% ee, about or at 75% ee, about or at least about 80% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all subranges and values therebetween. In some embodiments, the process for the preparation of (R)-3,4-methylenedioxymethamphetamine, the process provides (R)-3,4-methylenedioxymethamphetamine, or a pharmaceutically acceptable salt in an enantiomeric excess of a least 99.5%. In some embodiments, ee is measured by chiral HPLC.

In some embodiments of the process for the preparation of (R)-3,4-methylenedioxymethamphetamine, the (R)-3,4-methylenedioxymethamphetamine, or a pharmaceutically acceptable salt thereof has a chemical purity of greater than 95%, greater than 98%, or greater than 99% by HPLC.

In some embodiments, the present disclosure provides (R)-3,4-methylenedioxymethamphetamine, or a pharmaceutically acceptable salt prepared by a process described herein.

### Numbered Embodiments

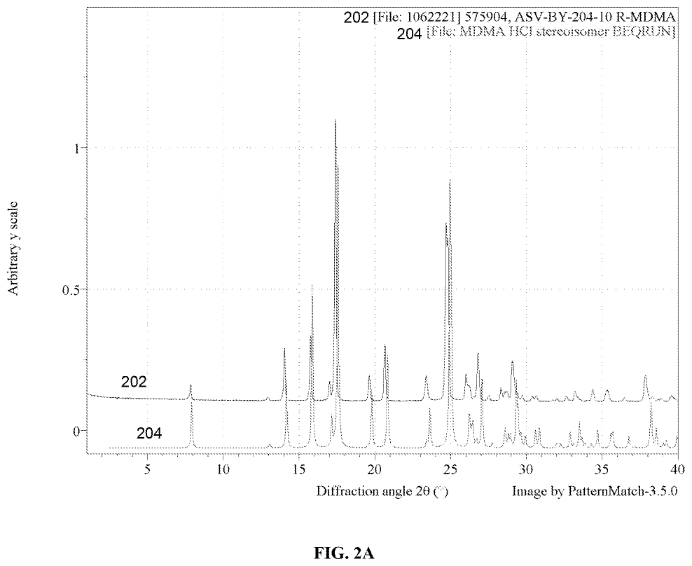

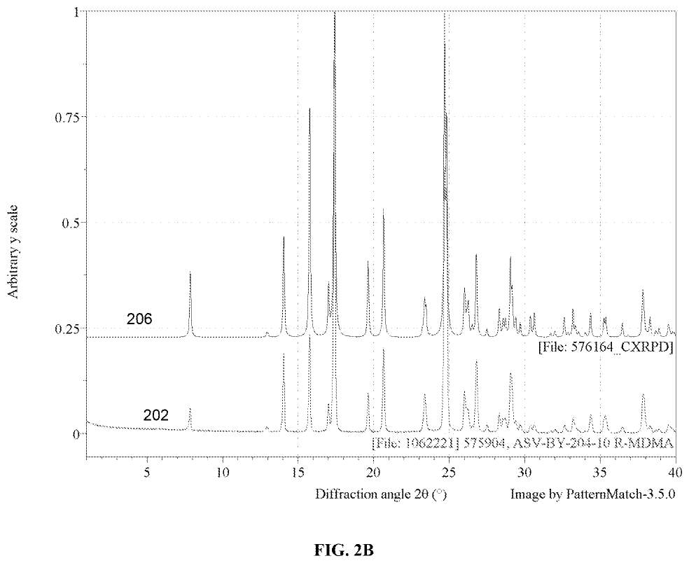

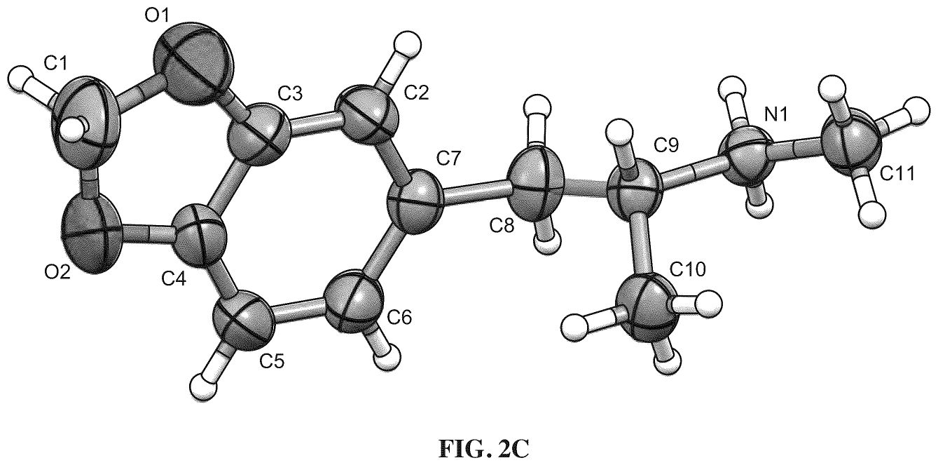

1. (R)-3,4-methylenedioxymethamphetamine HCl Form 1.
2. The composition of embodiment 1, wherein (R)-3,4-methylenedioxymethamphetamine HCl Form I is characterized by an X-ray Powder Diffraction (XPRD) pattern substantially similar to 204 in FIG. 2A and FIG. 2B.
3. The composition of any one of embodiments 1-2, wherein (R)-3,4-methylenedioxymethamphetamine HCl Form I is characterized by peaks in an XPRD pattern at 17.5±0.2, 24.8±0.2, and 15.8±0.2.
4. The composition of any one of embodiments 1-3, wherein (R)-3,4-methylenedioxymethamphetamine HCl Form I is characterized by peaks in an XPRD pattern at 17.5±0.2, 24.8±0.2, 15.8±0.2, 20.5±0.2, 14.0±0.2, 26.7±0.2, 29.0±0.2, 37.9±0.2, 7.9±0.2° 2θ.
5. The composition of any one of embodiments 1-4, wherein (R)-3,4-methylenedioxymethamphetamine HCl Form I exhibits a monoclinic crystal system with cell parameters of a=7.17±0.05 Å, b=7.61±0.05 Å, c=11.75±0.05 Å, α=90±1°, β=107±1°, γ=90±1°, V=612±5 Å³/cell.
6. The composition of any one of embodiments 1-5, wherein (R)-3,4-methylenedioxymethamphetamine HCl Form I exhibits a solubility of >110 mg/mL in water.
7. The composition of any one of embodiments 1-6, wherein (R)-3,4-methylenedioxymethamphetamine HCl Form I exhibits a Differential Scanning calorimetry (DSC) thermogram comprising an endotherm peak with an onset at 185° C.
8. The composition of any one of embodiments 1-7, wherein (R)-3,4-methylenedioxymethamphetamine HCl Form I exhibits a DSC thermogram comprising an endothermic peak at 186° C.
9. The composition of any one of embodiments 1-8, wherein (R)-3,4-methylenedioxymethamphetamine HCl Form I exhibits 0.2±0.1% weight loss between 52-231° C. as measured by thermogravimetric (TGA) analysis.
10. The composition of any one of embodiments 1-9, wherein (R)-3,4-methylenedioxymethamphetamine HCl Form I exhibits 5.28±0.02% weight gain from 5-95% relative humidity (RH) and a 5.35±0.02% weight loss from 95-5% RH at 10% RH increments under a nitrogen purge as measured by Dynamic Vapor Sorption (DVS).
11. The composition of any one of embodiments 1-10, wherein (R)-3,4-methylenedioxymethamphetamine HCl Form I exhibits physical stability upon stressing at 97% RH for 6 days.
12. A process for the preparation of (R)-3,4-methylenedioxymethamphetamine HCl Form 1, the process comprising:
   i) reacting an organometallic reagent prepared from a compound of Formula (I):

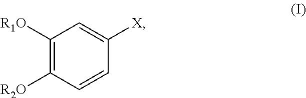

with a compound of Formula (IIa):

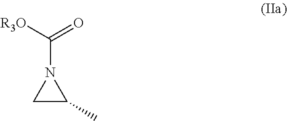

wherein R₃ is alkyl; and
   ii) converting the product of step i) to (R)-3,4-methylenedioxymethamphetamine, wherein X is a halogen; R₁ is a protecting group, R₂ is a protecting group or R₁ and R₂ together with the atoms to which they are attached form a 5-membered heterocycle.
13. The process of embodiment 12, wherein the compound of Formula (I) is a compound of Formula (Ia):

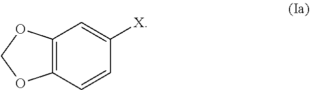

14. The process of any one of embodiments 12-13, wherein X is bromine.
15. The process of any one of embodiments 12-14, wherein R₃ is tert-butyl.
16. The process of any one of embodiments 12-15, wherein step i) comprises reacting the compound of Formula (I) or Formula (Ia) with magnesium.
17. The process of embodiment 16, wherein the step i) further comprises adding a copper (I) salt (e.g., CuI) to the reaction mixture.
18. The process of any one of embodiments 12-17, wherein the product of step i) is a compound of Formula (IIIb):

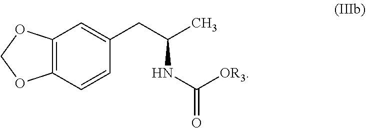

19. The process of any one of embodiments 12-17, wherein the product of step i) is a compound of Formula (IIIb′):

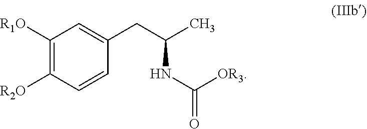

20. The process of any one of embodiments 12-19, wherein the step ii) comprises reacting a group of Formula (IVa) with a reducing agent to provide a group of Formula (Va), or a pharmaceutically acceptable salt thereof:

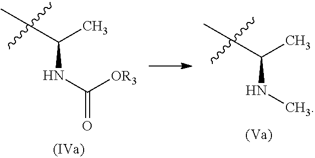

21. The process of embodiment 20, wherein the reducing agent is lithium aluminum hydride.
22. The process of any one of embodiments 12-21, wherein (R)-3,4-methylenedioxymethamphetamine HCl Form 1 is in an enantiomeric excess of a least 99.5%.
23. (R)-3,4-methylenedioxymethamphetamine HCl Form 1 prepared by a process of any one of embodiments 12-22.

### EXAMPLES

Compounds of the present disclosure are synthesized using the following exemplary methods or other methods that are known to those skilled in the art.

General reaction conditions are provided, and reaction products are purified by known methods including silica gel chromatography using various organic solvents such as hexane, dichloromethane, ethyl acetate, methanol and the like or preparative reverse phase high pressure liquid chromatography.

Preparation of compounds involves the protection and deprotection of various chemical groups. The need for protection and deprotection, and the selection of appropriate protecting groups is readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

### General Synthesis of (R)-3,4-methylenedioxymethamphetamine (MDMA)

(R)-3,4-methylenedioxymethamphetamine (MDMA) can be synthesized according to Scheme 1, below.

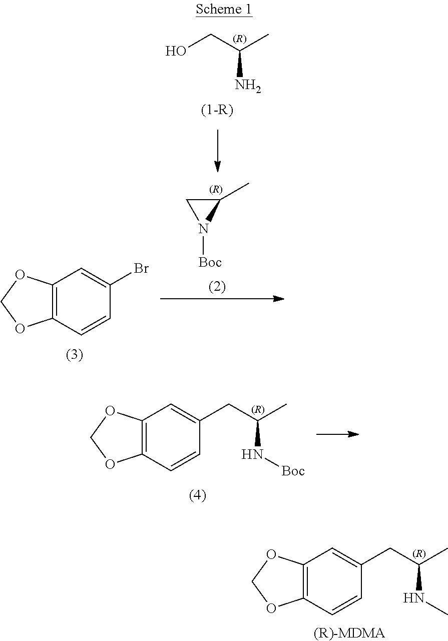

### Step 1: Synthesis of tert-butyl (R)-2-methylaziridine-1-carboxylate (2)

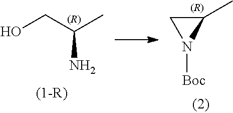

To D-alaninol (1) is added 4-toluenesulfonyl chloride (TsCl), followed by potassium hydroxide (KOH), which installs the tosyl group on the alcohol and deprotonates the nitrogen, facilitating aziridine formation. The enantiopure aziridine intermediate is subsequently protected with di-tert-butyl dicarbonate (Boc anhydride) in organic solvent (e.g., halogenated solvent such as dichloromethane (DCM), either solvent such as tetrahydrofuran (THF) or methyl-tetrahydrofuran (Me-THF)) to form compound (2), which is then purified. In the scheme above, compound (2) may be synthesized as shown or it may be purchased since compound (2) is a commercially available compound.

### Step 2: Synthesis of tert-butyl (R)-(1-(benzo[d][1,3]dioxol-5-yl)propan-2-yl)carbamate (4)

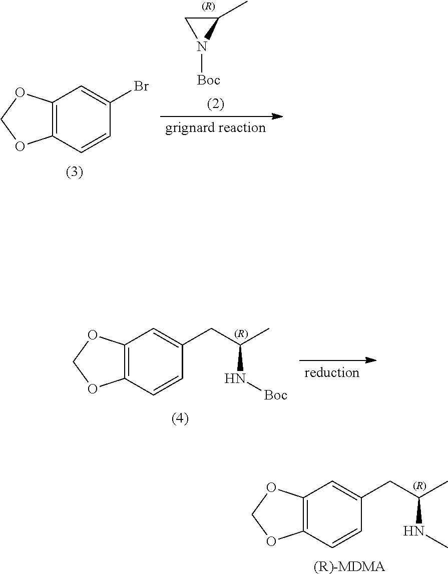

In a second step, the Grignard reagent of 5-bromobenzo[d][1,3]dioxole (3) is generated by treatment of 5-bromobenzo[d][1,3]dioxole (3) with magnesium, optionally in the presence of I₂, in organic solvent (e.g., ether solvent such as tetrahydrofuran (THF) or methyl-tetrahydrofuran (Me-THF)) and the mixture heated. After formation of the Grignard species, the solution is cooled and Copper (I) salt e.g., CuI or CuBr.SMe₂ is added. The previously isolated Boc-aziridine (2) is then added to the cooled solution of cuprate and stirred until the reaction is complete. The reaction is then quenched with an aqueous solution e.g., with NH₄Cl and the organic phase extracted with organic solvent and purified by chromatography on SiO₂, or by crystallization.

### Step 3: Synthesis of (R)-3,4-methylenedioxymethamphetamine (MDMA)

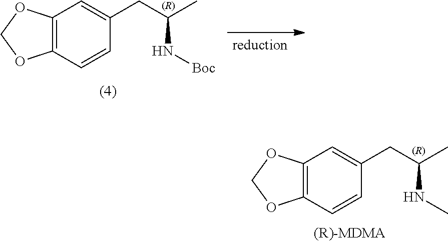

The Boc-MDA (4) is then dissolved in organic solvent (e.g., in an ether solvent such as tetrahydrofuran, (THF) or 2-methyltetrahydrofuran (Me-THF)), cooled and treated with lithium aluminum hydride (LiAlH4) to reduce the carbamate to the methylamine. In some embodiments, the solution is heated (e.g., to reflux) to reduce the carbamate to the methylamine. The reaction is quenched and worked-up via the Fieser method with water, NaOH and methyl tert-butyl ether. The crude MDMA free base can then be isolated e.g., by crystallization, or converted into the HCl salt using a solution of HCl.

(S)-3,4-methylenedioxymethamphetamine (MDMA) synthesized according to Scheme 1 employing L-alaninol instead of D-alaninol.

### Example 1: Synthesis of (R)-3,4-methylenedioxymethamphetamine (MDMA)

(R)-3,4-methylenedioxymethamphetamine (MDMA) can be synthesized according to the Scheme below.

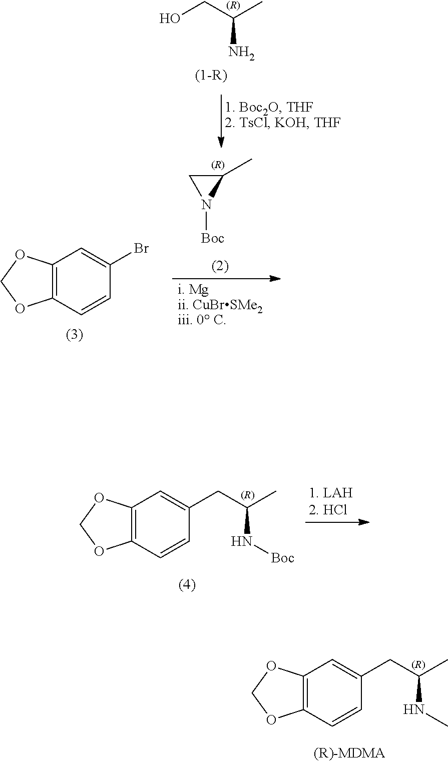

### Step 1, Route 1: Synthesis of tert-butyl (R)-2-methylaziridine-1-carboxylate

To a 300 mL jacketed reactor was charged D-alaninol (20 g, 266.3 mmol, 1.00 eq) and DCM (100 mL) then cooled to 0-5° C. under N₂. A solution of Boc₂O (64.2 g, 292.9 mmol, 1.1 eq) in DCM (100 mL) was added over 40 minutes maintaining an internal temperature of <12° C. After the addition was complete the reaction was warmed to 20° C. After 3.5 hours no D-alaninol was detected by TLC. The reaction was diluted with water (50 mL), then the organic layer was washed with sat. NaCl (2×50 mL), dried over MgSO₄, filtered and concentrated under reduced pressure to afford crude Boc-D-alaninol (62.2 g). The crude product was charged to the 300 mL reactor as a solution in THF (200 mL) at 20° C. Solid TsCl (66.2 g, 1.3 eq) was charged resulting in a 10° C. endotherm. Solid powdered KOH (60.1 g, 4.0 eq) was added in portions—the initial KOH charge resulted in a strong exotherm (>20° C.), requiring setting the TCU to 0° C. Subsequent charges only saw <5° C. exotherms. As the mixture cooled to <30° C. the reaction was held at 20° C. After 20 minutes the mixture became very thick and a further 100 mL of THF was added to aid mixing. After stirring for 21 hours the mixture was filtered and the filter cake washed with MTBE. The filtrate was concentrated under reduced pressure to afford crude compound 2 (52.1 g). Chromatographic purification on silica (600 g), eluting with 0-60% EtOAc/heptanes afforded compound 2 (13.1 g, 31%) as a light yellow oil.

### Step 1, Route 2: Synthesis of tert-butyl (R)-2-methylaziridine-1-carboxylate

To a 250 mL RBF was charged D-alaninol (EMP-2, 5.0 g, 66.6 mmol, 1.00 eq) and THF (33 mL) then cooled to 0-5° C. under N₂. A solution of Boc₂O (14.9 g, 67.9 mmol, 1.02 eq) in THF (7.5 mL) was added over 50 minutes maintaining an internal temperature of <10° C. After the addition was complete the reaction was warmed to 15° C. After 16 hours no D-alaninol was detected by TLC. A solution of TsCl (17.8 g, 1.4 eq) in THF (10 mL) was added at 15° C., resulting in a 3° C. endotherm, followed by a THF (2.5 mL) rinse. Solid powdered KOH (18.3 g, 4.9 eq) was added resulting in a strong exotherm (>35° C.), requiring external cooling. The reaction mixture became white slurry. After stirring for 22 hours the mixture was diluted with MTBE (25 mL, 5 vol), filtered and the filter cake washed with MTBE. The filtrate was passed through a silica plug (30 g), eluting with MTBE to afford compound 2 (7.37 g, 57%,).

### Step 2: Synthesis of tert-butyl (R)-(1-(benzo[d][1,3]dioxol-5-yl)propan-2-yl)carbamate (4)

To a 100 mL RBF was charged Mg (0.155 g, 6.36 mmol, 2.00 eq), I₂ (one crystal) and THF (3 mL) at room temperature under N₂. Bromide 3 (0.1 g) was added to the mixture and heated to 50° C. at which time the iodine color disappeared and the internal temperature rose to 56° C. Bromide 3 (1.20 g, total added 1.30 g, 6.46 mmol, 2.0 eq) was added, via syringe, to the mixture dropwise maintaining an internal temperature of 45-55° C. over 10 minutes. After addition was complete the syringe was rinsed with THF (0.5 mL) and the rinse charged to the reaction at 49° C. After stirring for 1.5 hours the batch was a clear amber color with an internal temperature of 19.6° C. THF (2.0 mL) was added. The flask was cooled to 0.8° C. using an ice/water bath then solid CuBr.SMe₂ (0.131 g, 0.636 mmol, 0.20 eq) was charged in one portion. An exotherm to 6° C. was observed. After cooling to 0.5° C. a solution of R-Boc-aziridine 2 (87.8 wt %,, 0.57 g, 3.18 mmol, 1.00 eq) in THF (1.5 mL) was added over 20 minutes, while maintaining an internal temperature <6° C. After stirring for 4 hours TLC analysis (5:1 heptanes/EA) of the brown slurry showed complete reaction. After a further 20 minutes the reaction was quenched with dropwise addition sat. NH₄Cl (5.0 mL), while maintaining an internal temperature <18° C. (3 minutes). After stirring for 12 minutes at room temperature the biphasic mixture was diluted with EtOAc (3 mL). The layers were separated, and the aqueous layer was extracted with EtOAc (2×3 mL). The combined organic layers dried over Na₂SO₄ (2.5 g), filtered and concentrated under reduced pressure. Chromatographic purification in silica, eluting with 0-15% EtOAc/heptanes afforded compound 4 (0.77 g, 87%,) as a solid.

### Step 3: Synthesis of (R)-3,4-methylenedioxymethamphetamine (MDMA)

To a 5-neck 2 L jacketed reactor was charged compound 4 (78.3 g, 280.6 mmols, 1.0 eq) and THF (1.4 L, 18 vol). The solution is heated to 55 to 60° C. and a solution of lithium aluminum hydride (LAH, 2 M in THF, 430 mL, 842 mmols, 3.0 eq) is slowly added over 1.5 hours, maintaining an internal temperature of 55 to 65° C. After 1 hour, IPC analysis by HPLC showed 0.6% compound 4 remaining. After an additional 2 hours, IPC analysis showed 0.5% compound 4 remaining. The batch was cooled to 0 to 5° C. and 32 mL of water was slowly charged over ˜80 minutes, keeping the reaction temperature below 15° C. 15% NaOH (32 mL) was then added over 8 minutes, followed by water (96 mL). The thick slurry was warmed to ambient temperature and stirred overnight. The slurry was filtered, and the filter cake washed with methyl tert-butyl ether (2×2 vol). The filtrate was concentrated under reduced pressure to afford crude (R)-MDMA (54.0 g) which was diluted with MeOH (118 mL) and MTBE (1.3 L) at ambient temperature under nitrogen. To the solution was added 3M HCl in MeOH (140 mL, 1.5 eq), resulting in the formation of a white slurry. The slurry was stirred for 30 minutes and filtered, and the filter cake was washed with MTBE (3×200 mL). The cake was dried under reduced pressure to afford (R)-MDMA-HCl (55.2 g, 85%) with an HPLC purity of 99.5% area by HPLC.

### Example 2: Characterization of (R)-3,4-methylenedioxymethamphetamine (MDMA) HCl Crystalline Solid

(R)-3,4-methylenedioxymethamphetamine (MDMA) HCl characterized in this example was synthesized using the method described in Example 1. A sample of this crystalline material was characterized using the various techniques further discussed herein and designated Form 1. Table 1 below is a summary of the characterization results of the (R)-MDMA HCl sample.

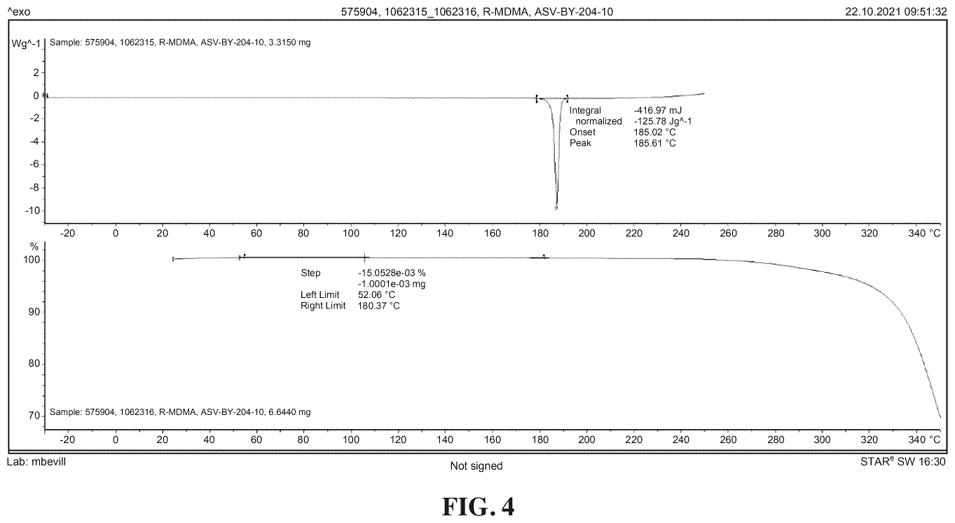

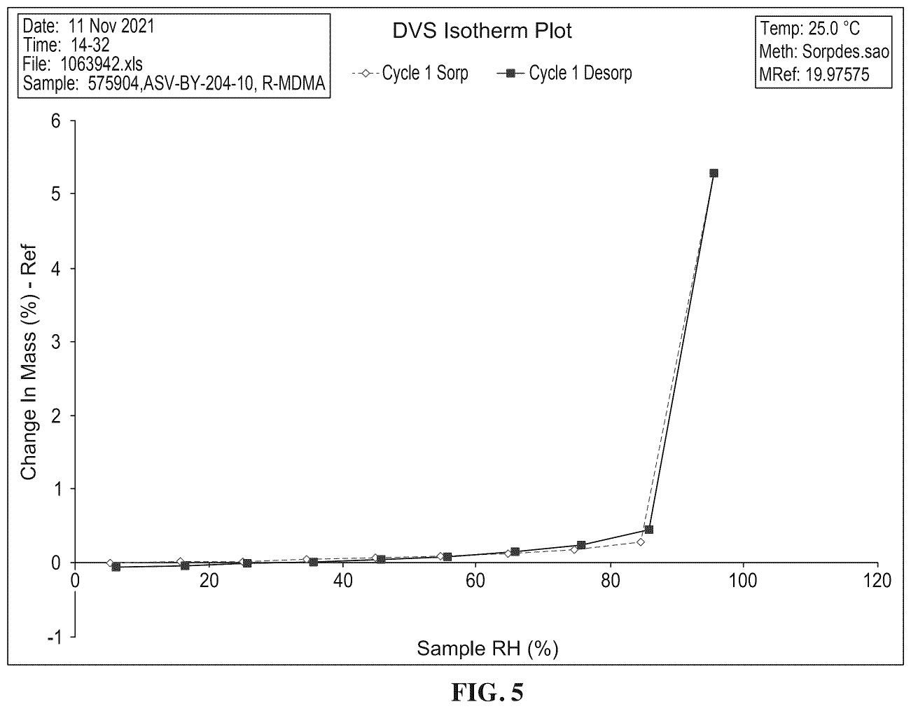

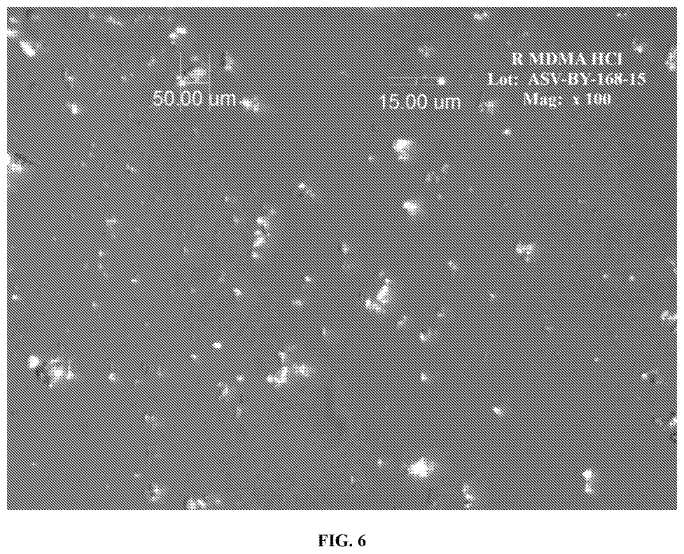

TABLE 1
Summary of Characterization Results

| Technique | Results | | | |
|---|---|---|---|---|
| XRPD | Anhydrous, unsolvated crystalline form designated Form I and | | | |
| | successfully indexed. See Table 2 for cell parameters. | | | |
| | The single crystal structure for Form I was solved from a sample | | | |
| | generated during the form screen and unit cell parameters were | | | |
| | found to be consistent with SCXRD. | | | |
| | FIG. 2A: XRPD overlay of Form I patterns (experimental 202 | | | |
| | vs literature 204) | | | |
| | FIG. 2B: XRPD overlay of calculated 206 and experimental | | | |
| | 202 patterns | | | |
| | FIG. 2C: Atomic displacement ellipsoid diagram of R- | | | |
| | MDMA•HCl | | | |
| | Table 2: Crystal data and data collection parameters for R- | | | |
| | MDMA HCl Form I | | | |
| Approximate | The compound exhibited high solubility in aqueous environments, | | | |
| solubility at RT | as well as good solubility in ACN, DCM, and most alcohols. | | | |
| | Lower solubility was observed in most organic solvents tested. | | | |
| | pH of a 10 mg/mL aqueous solution is approximately 6 using pH test | | | |
| | strips | | | |
| | Table 3: Approximate Solubilities in various solvents at Ambient | | | |
| | Temperature | | | |
| ¹H NMR | Consistent w/chemical structure, negligible solvent | | | |
| | FIG. 3A-B: Proton NMR of R-MDMA HCl | | | |
| DSC | Sharp endotherm at 186° C. (onset 185° C.) corresponds with the melt. | | | |
| TGA | 0.2% weight loss 52-231° C. (very little weight loss consistent with | | | |
| (Collected at ramp | an anhydrous, unsolvated material) | | | |
| rate of 10° C./min) | FIG. 4: DSC and TGA thermograms of R-MDMA HCl Form I | | | |
| DVS | 5.28% weight gain 5-95% RH: | | | |
| | 0.28% wt. gain 5-85% RH | | | |
| | 5.00% wt. gain 85-95% RH (eq. to 0.7 mol water) | | | |
| | 5.35% weight loss 95-5% RH: | | | |
| | 4.84% wt. loss 95-85% RH | | | |
| | 0.51% wt. loss 85-5% RH | | | |
| | Post-DVS crystalline form was found to be Form I | | | |
| | FIG. 5: DVS isotherm for R-MDMA HCl Form I | | | |
| Optical Microscopy | See FIG. 6 | | | |
| | PSD | d10 (μm) | d50 (μm) | d90 (μm) |
| Particle size | Form I | 5.10 | 19.2 | 48.8 |
| distribution | Form IV | 4.25 | 11.2 | 25.1 |

Notes:
Temperatures (° C.) reported for DSC data are transition maxima unless otherwise stated.

### X-Ray Powder Diffraction (XRPD)

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence.

Diffraction patterns were collected using a scanning position-sensitive detector (X'Celerator) located 240 mm from the specimen and Data Collector software v. 5.5. Calculated XRPD patterns were generated for Cu radiation using Mercury and the atomic coordinates, space group, and unit cell parameters from the single crystal structure.

An XRPD pattern 202 of R-MDMA HCl Form 1 as-received from Example 1 is shown in FIG. 2A. A single crystal of R-MDMA HCl Form I was grown from a solution in DCM, and the structure was solved. The crystal system is monoclinic and the space group is P21. The cell parameters and calculated volume are: a=7.1660(2) Å, b=7.6076(3) Å, c=11.7505(3) Å, α=90°, β=107.121(3)°, γ=90°, V=612.20(3) Å3. The formula weight is 229.70 g mol−1 with Z=2, resulting in a calculated density of 1.246 g cm−3. Further details of the crystal data and crystallographic data collection parameters are summarized in Table 2. An atomic displacement ellipsoid drawing of R-MDMA HCl Form I is shown in FIG. 2C. An XRPD pattern calculated from the single crystal data 206 is consistent with the experimental pattern 202 for R-MDMA HCl Form I, illustrated in FIG. 2B. The asymmetric unit shown in FIG. 2C contains one R-MDMA cation and one chloride anion.

TABLE 2
Crystal Data and Data Collection
Parameters of R-MDMA HCl Form I

| | |
|---|---|
| Empirical formula | C₁₁H₁₆ClNO₂ |
| Formula weight (g mol⁻¹) | 229.70 |
| Temperature (K) | 298(2) |
| Wavelength (Å) | 1.54184 |
| Crystal system | monoclinic |
| Space group | P2₁ |
| Unit cell parameters | |
| a = 7.1660(2) Å | α = 90° |
| b = 7.6076(3) Å | β = 107.121(3)° |
| c = 11.7505(3) Å | γ = 90° |
| Unit cell volume (Å³) | 612.20(3) |
| Cell formula units, Z | 2 |
| Calculated density (g cm⁻³) | 1.246 |
| Absorption coefficient (mm⁻¹) | 2.620 |
| F(000) | 244 |
| Crystal size (mm³) | 0.2 × 0.14 × 0.03 |
| Reflections used for cell | 4718 |
| measurement | |
| θ range for cell measurement | 6.4220°-75.6220° |
| Total reflections collected | 5739 |
| Index ranges | −8 ≤ h ≤ 8; −9 ≤ k ≤ 8; −11 ≤ |
| | l ≤ 14 |
| θ range for data collection | θₘᵢₙ = 3.936°, θₘₐₓ = 75.709° |
| Completeness to θₘₐₓ | 97.3% |
| Completeness to θ_{full} = | 100% |
| 67.684° | |
| Absorption correction | multi-scan |
| Transmission coefficient | 0.577-1.000 |
| range | |
| Refinement method | full matrix least-squares on F² |
| Independent reflections | 2129 [Rᵢₙₜ = 0.0350, R_{σ} = 0.0293] |
| Reflections [I > 2σ(I)] | 2014 |
| Reflections/restraints/ | 2129/1/147 |
| parameters | |
| Goodness-of-fit on F² | S = 1.07 |
| Final residuals [I > 2σ(I)] | R = 0.0313, R_{w} = 0.0873 |
| Final residuals [all reflections] | R = 0.0327, R_{w} = 0.0886 |
| Largest diff. peak and hole (e | 0.169, −0.272 |
| Å⁻³) | |
| Max/mean shift/standard | 0.000/0.000 |
| uncertainty | |
| Absolute structure | Flack parameter: −0.002(13) |
| determination | |

### Solubility of R-MDMA HCl Form 1 in Various Solvents

Aliquots of various solvents were added to measured amounts of a Form I with agitation (typically sonication) at ambient temperature until complete dissolution was achieved, as judged by visual observation. If dissolution occurred after the addition of the first aliquot, values are reported as “>”. If dissolution did not occur, values are reported as “<”. The solubility values for various solvents are reported in Table 3 below.

TABLE 3
Approximate Solubilities in Various
Solvents at Ambient Temperature

| No. | Solvent | Solubility (mg/mL)³ |
|---|---|---|
| 1 | MeOH | >114 |
| 2 | Water | >110 |
| 3 | 90:10 Acetone/Water | >76 |
| 4 | EtOH | 25 |
| 5 | DCM | 16 |
| 6 | CAN | 7 |
| 7 | IPA | 6 |
| 8 | MeOH/MTBE (20:80) | 3 |
| 9 | 1-BuOH | 3 |
| 10 | MeOH/MTBE (10:90) | <2 |
| 11 | t-BuOH | 1 |
| 12 | Acetone | <1 |
| 13 | p-dioxane | <1 |
| 14 | EtOAc | <1 |
| 15 | Heptane | <1 |
| 16 | MeOH/MTBE (5:95) | <1 |
| 17 | MTBE | <1 |
| 18 | THF | <1 |

Notes:
Solubilities are calculated based on the total solvent used to give a solution; actual solubilities may be greater because of the volume of the solvent portions used or a slow rate of dissolution. Values are rounded to the nearest whole number. If dissolution did not occur as determined by visual assessment, the value is reported as “<”. If dissolution occurred as determined by the visual assessment after the addition of the first aliquot, the value is reported as “>”.

The compound exhibited high solubility in aqueous environments, as well as good solubility in ACN, DCM, and most alcohols. Lower solubility was observed in most organic solvents tested.

### Proton Nuclear Magnetic Resonance (¹H NMR) Spectroscopy

FIG. 3B shows a ¹H NMR spectrum for (R)-MDM HCl Form I. The solution NMR spectra was acquired with an Avance 600 MHz spectrometer. The samples were prepared by dissolving approximately 5-10 mg of sample in DMSO-d6 containing TMS. Data acquisition parameters are shown in FIG. 3A. The ¹H NMR spectrum is consistent with the chemical structure of R-MDMA HCl with negligible solvent present.

### Thermogravimetric and Differential Scanning calorimetry

Thermogravimetric and Differential Scanning calorimetry analysis was performed on (R)-MDMA HCl Form I and is shown in FIG. 4. The DSC data shows a sharp endothermic peak at 186° C. with an onset at 185° C. that corresponds to a melt of the sample. The TGA data shows 0.2% weight loss between 52-231° C. The minimal weight loss is consistent with an anhydrous and unsolvated material.

Thermogravimetric and Differential Scanning calorimetry analyses were performed using a Mettler-Toledo TGA/DSC3+ analyzer. Temperature and enthalpy adjustments were performed using indium, tin, zinc, and phenyl salicylate, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. Samples were analyzed from 25° C. to 350° C. at 10° C./min.

### Dynamic Vapor Sorption (DVS)

Dynamic vapor sorption data were collected on a Surface Measurement System DVS Intrinsic instrument. The DVS sorption data for (R)-MDMA HCl Form I is shown in FIG. 5. (R)-MDMA HCl Form I exhibits low hygroscopicity between 5% and 85% RH, picking up only 0.28 wt % water vapor (or 0.04 moles water). Significant hygroscopicity is then observed between 85% and 95% RH, with a weight gain of 5.00% corresponding with the sorption of 0.7 moles of water per mole of salt. It should be noted that the maximum equilibration time was reached at 95% RH, indicating that additional sorption likely would have occurred if the sample had remained at 95% RH for a longer duration. All of the weight was lost on desorption with virtually no hysteresis. The post-DVS solids were consistent with Form I by XRPD.

Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### Optical and Polarized Light Microscopy

Throughout the salt screen, samples were observed under a Motic or Wolfe optical microscope with crossed polarizers or under a Leica stereomicroscope with a first order red compensator with crossed polarizers. FIG. 6 shows an image of (R)-MDMA HCl Form I at 100× magnification.

Polarized light microscopy for select forms was performed using a Leica DM LP microscope equipped with a SPOT Insight™ color digital camera. Each sample was placed on a glass slide, a cover glass was placed over the sample, and a drop of mineral oil was added to cover the sample by capillarity. Each sample was observed with crossed polarizers and a first order red compensator. Objectives used are listed in the images. Images were captured using SPOT software (v. 4.5.9). A micron bar was inserted onto each image as a reference for particle size.

### Conclusion

Only Form I was identified upon characterization of the R-MDMA HCl material produced by the method of Example 1. In order to identify other potential polymorphic forms of R-MDMA HCl, a stable form and polymorph screen was conducted as described further in Example 3.

### Example 3: Stable Form and Polymorph Screen

Twenty-nine stable form and polymorph screen experiments were conducted on R-MDMA HCl Form I which was synthesized by the method described in Example 1. These screen experiments include slurries, evaporations, vapor diffusions, RH stressing, solvent/antisolvent, and heating experiments starting with Form I and are described in the Tables below. Note: (a) Solvent ratios are volume/volume, (b) times and temperatures are approximate and (c) solids from RT slurries were analyzed while damp. “PO” denotes preferred orientation. “B/E” denotes birefringence and extinction.

### Milling

A dry milling experiment was conducted to investigate the effect on crystallinity. R-MDMA HCl Form I was milled at 30 Hz for 3×10 minutes resulting in Form I with no apparent decrease in crystallinity, as determined by XRPD. The solids were observed to be clumped fines and aggregates and exhibited birefringence.

For the following experiments described in Tables 4-12, the state of the (R)-MDMA HCl material was determined by XRPD unless stated otherwise.

TABLE 4
Slow Evaporation

| Solvent | Observations | State |
|---|---|---|
| ACN | white solids, spheroidal overlapping needles, B/E | Form I |
| DCM | white solids, overlapping needles, B/E | Form I |

TABLE 5
Crash Cooling

| Solvent | Conditions | Observations | State |
|---|---|---|---|
| ACN | from 75° C. to freezer, | white aggregate needles, | Form I |
| | 5 hours | B/E | |
| EtOH | 1) from 75° C. to freezer | 1) small aggregates, B/E | Form I, |
| | 2) sit in freezer, 4 days | 2) white solids, clumped | PO |
| | | aggregates, B/E | |

TABLE 6
Slow Cooling

| Solvent | Conditions | Observations | State |
|---|---|---|---|
| ACN | from 75° C. to RT, 5 hours | white thick prisms, B/E | Form I |
| EtOH | from 75° C. to RT, 5 hours | thin plates and | Form I* |
| | | aggregates, B/E | |

*Form determined by indexing on single crystal diffractometer. No data was collected.

TABLE 7
Vapor Diffusion

| Solvent | Conditions | Observations | State |
|---|---|---|---|
| EtOH | w/MTBE, 4 | white solids, fused aggregates | Form I, PO |
| | days | and tabular plates, B/E | |
| MeOH | w/Et₂O, 4 | white solids, fused aggregates | Form I, PO |
| | days | and prisms, B/E | |

TABLE 8
Fast Evaporation

| Solvent | Observations | State |
|---|---|---|
| IPA | clear sticky oil that crystalized upon agitation, needles | Form I |
| | and aggregates, B/E | |
| MeOH | white solids, spheroidal needles and aggregates, B/E | Form I |

TABLE 9
Solvent/Antisolvent

| Solvent | Conditions | Observations | State |
|---|---|---|---|
| MeOH | S/AS w/MTBE | white ppt. rapidly crashed out | Form I, |
| | 1:4 | upon 4^{th} aliquot addition, fines, B/E | PO |

TABLE 10
Stable Form screen from Slurries in
Organic Solvent at Room Temperature

| Solvent | Conditions | Observations | State |
|---|---|---|---|
| ACN | RT, 14 d | white fines and aggregates, B/E | Form I |
| 1-BuOH | RT, 14 d | white fines and aggregates, B/E | Form I |
| t-BuOH | ~75° C., 7 d | off-white solids, dendrites and | Form I |
| | | aggregates, B/E | |
| DCM | RT, 14 d | white fines and aggregates, B/E | Form I |
| 90:10 EtOAc/ | RT, 14 d | white fines and aggregates, B/E | Form I |
| MeOH | | | |
| EtOH | RT, 14 d | white fines and aggregates, B/E | Form I |
| IPA | RT, 14 d | white fines and aggregates, B/E | Form I |
| | ~5° C., 14 d | white fines and aggregates, B/E | Form I |
| MeOH/MTBE | RT, 5 d | white solids, fines, B/E | Form I |
| (10:90) | | | |

TABLE 11
Stable Form Screen from Slurries in Aqueous/Organic
Mixtures at Room Temperature for 7 Days

| Solvent | Observations | State |
|---|---|---|
| THF/water 98:2 (a_{w} 0.38) | white solids, fines, B/E | Form I |
| acetone/water 98:2 (a_{w} 0.35) | white solids, fines, B/E | Form I |
| p-dioxane/water 95:5 (a_{w} | white solids, fines, B/E | Form I |
| 0.40) | | |

TABLE 12
Relative Humidity Stressing

| Conditions | Observations | State |
|---|---|---|
| 75% RH, 6 d | free-flowing solids, B/E | Form I |
| 90% RH, 6 d | mostly free-flowing solids, some clumps, | Form I |
| | B/E | |
| 97% RH, 6 d | off-white fines, B/E, some deliquesence | Form I, PO |

### Heating

An (R)-MDMA HCl Form 1 sample was heated by DSC from RT to 200° C. and cooled down to RT. XRPD analysis of the treated sample showed Form I crystalline state.

### Conclusion

XRPD analysis conducted on samples from the stable form and polymorph screen of Example 3 showed that Form I was formed in each case.

### Example 4: Amorphous Form Preparation

Attempts were made to prepare amorphous material of (R)-MDMA HCl by rotary evaporation and lyophilization as summarized in Table 13 below. Form I crystalline material of (R)-MDMA HCl was obtained in each case and verified by XRPD.

TABLE 13
Amorphous Material Attempts

| Conditions | Observations | State |
|---|---|---|
| Water, lyophilize, 1 d | off-white solids | Form I |
| MeOH, unfiltered, rotovap | off-white flakey solids, crystalline | Form I |
| | solids on edge of vial, B/E | |
| DCM, filtered, rotovap | white solids, fines, B/E | Form I |

Since the attempt to prepare amorphous material of (R)-MDMA HCl by lyophilization was unsuccessful, cycling DSC experiments shown in Table 14 were performed on (R)-MDMA HCl Form I in an attempt to determine a glass transition temperature (Tg).

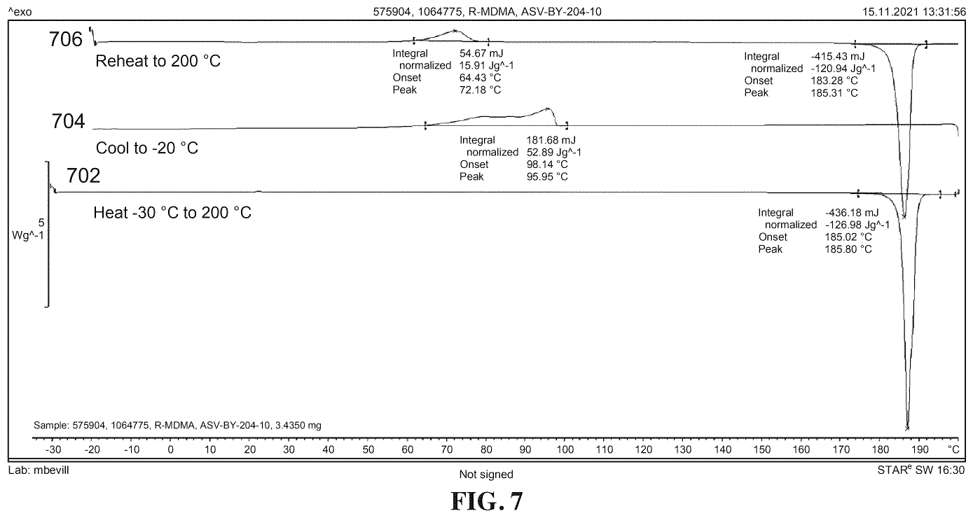

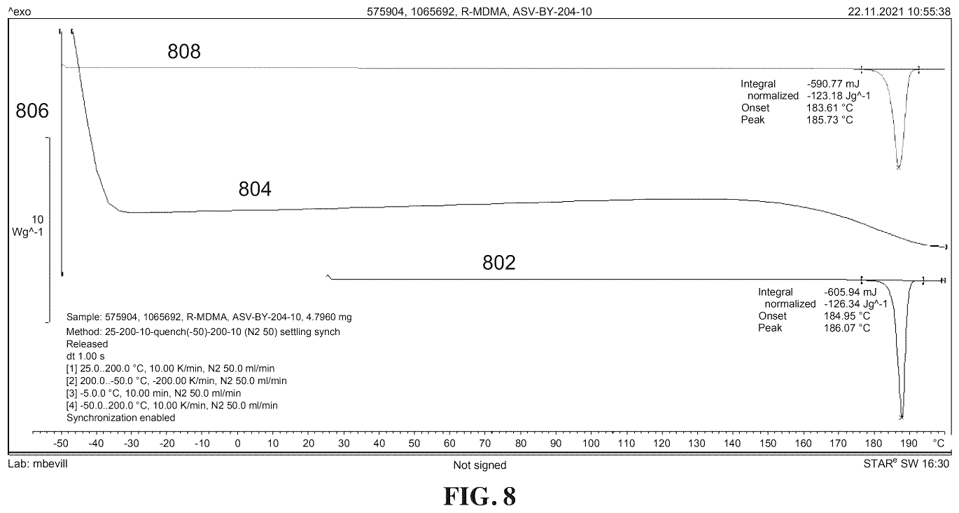

TABLE 14
Cycling DSC Experiments

| Technique | Results |
|---|---|
| cycling DSC | 1) heat −30° C. to 200° C.: endo onset 185° C. |
| (10K/min on | 2) cool to −20° C.: broad exo 96° C. (peak max) |
| all steps), | 3) reheat to 200° C.: exo 72° C. (peak max), endo |
| FIG. 7 | onset 183° C. |
| cycling DSC | 1) heat RT to 200° C.: endo onset 185° C. |
| (200K/min | 2) cool to −50° C. (200 K/min): significant drift |
| cooling step), | 3) hold at −50° C., 10 min: no events observed |
| FIG. 8 | 4) reheat to 200° C.: endo onset 184° C. |

Notes:
Temperatures (° C.) reported for DSC data are transition maxima unless otherwise stated.

FIG. 7 shows a cycling DSC thermogram for (R)-MDMA HCl Form 1. The material was initially heated past the melt to 200° C., curve 702, then cooled to −20° C., curve 704. A broad exothermic event on the cooling step (observed between ˜100° C. and 65° C.) is consistent with crystallization. Reheating of the material, curve 706, caused an additional exothermic event at 65° C., possibly corresponding with a form change, followed by a likely melt at 183° C. (onset), consistent with the melt onset for Form I.

FIG. 8 shows an additional cycling DSC experiment performed on Form I in an effort to determine Tg. The sample was initially heated past the melt to 200° C., curve 802, then was cooled to −50° C. at a rate of 200 K/min, curve 804. The material was held at −50° C. for 10 minutes, curve 806, before being reheated to 200° C., curve 808. A sharp endotherm at 184° C. (onset) in the final heating step is consistent with the melt onset for Form I. Despite the rapid cooling rate, the melted material crystallized, illustrating a strong tendency for amorphous material to crystallize.

### Conclusion

All attempts to generate amorphous material, including lyophilization and rotary evaporation, resulted in crystalline form (R)-MDMA HCl Form I. R-MDMA HCl Form I is a stable polymorph with a high melting point and aqueous solubility, making R-MDMA HCl Form 1 suitable for development as a pharmaceutical active ingredient.

1-23. (canceled)

24. A pharmaceutical composition comprising crystalline (R)-3,4-methylenedioxymethamphetamine ((R)-MDMA) HCl characterized by X-ray Powder Diffraction (XRPD) peaks at 7.9±0.2, 14.0±0.2, and 15.8±0.2° 2θ.

25. The pharmaceutical composition of claim 24, wherein the crystalline (R)-MDMA HCl is characterized by an XPRD pattern substantially similar to 204 in FIG. 2A.

26. The pharmaceutical composition of claim 24, wherein the crystalline (R)-MDMA HCl is further characterized by XRPD peaks at 17.5±0.2, 24.8±0.2, and 15.8±0.2° 2θ.

27. The pharmaceutical composition of claim 24, wherein the crystalline (R)-MDMA HCl is further characterized by XRPD peaks at 17.5±0.2, 24.8±0.2, 20.5±0.2, 26.7±0.2, 29.0±0.2, and 37.9±0.2° 2θ.

28. The pharmaceutical composition of claim 24, wherein the crystalline (R)-MDMA HCl exhibits a monoclinic crystal system with cell parameters of a=7.17±0.05 Å, b=7.61±0.05 Å, c=11.75±0.05 Å, α=90±1°, β=107±1°, γ=90±1°, V=612±5 Å³/cell.

29. The pharmaceutical composition of claim 24, wherein the crystalline (R)-MDMA HCl exhibits a solubility of >110 mg/mL in water.

30. The pharmaceutical composition of claim 24, wherein the crystalline (R)-MDMA HCl exhibits a Differential Scanning calorimetry (DSC) thermogram comprising an endotherm peak with an onset at 185° C.

31. The pharmaceutical composition of claim 24, wherein the crystalline (R)-MDMA HCl exhibits a DSC thermogram comprising an endothermic peak at 186° C.

32. The pharmaceutical composition of claim 24, wherein the crystalline (R)-MDMA HCl exhibits 0.2±0.1% weight loss between 52-231° C. as measured by thermogravimetric (TGA) analysis.

33. The pharmaceutical composition of claim 24, wherein the crystalline (R)-MDMA HCl exhibits 5.28±0.02% weight gain from 5-95% relative humidity (RH) and a 5.35±0.02% weight loss from 95-5% RH at 10% RH increments under a nitrogen purge as measured by Dynamic Vapor Sorption (DVS).

34. The pharmaceutical composition of claim 24, wherein the crystalline (R)-MDMA HCl exhibits physical stability upon storage at 97% RH for 6 days.

35. The pharmaceutical composition of claim 24, wherein the pharmaceutical composition is an oral dosage form.

36. The pharmaceutical composition of claim 24, where the pharmaceutical composition further comprises a pharmaceutically acceptable excipient.

37. The pharmaceutical composition of claim 24, wherein the enantiomeric excess of (R)-MDMA in the pharmaceutical composition is least about 95%.

38. The pharmaceutical composition of claim 24, wherein the enantiomeric excess of (R)-MDMA in the pharmaceutical composition is least about 98%.
